(19) United States
(12) Patent Application Publication
(10) Pub. No.: US 2009/0139522 A1
(43) Pub. Date: Jun. 4, 2009

(54) Monitor for automatic resuscitator with optional gas flow control

(75) Inventors: Glen Thomson, Citrus Heights, CA (US); James I-Che Lee, Sacramento, CA (US); Abdolreza Saied, Carmichael, CA (US); Matthew Fong, Sacramento, CA (US)

Correspondence Address: James Lee;VORTRAN Medical Technology 1, Inc., 21 Golden Land Court, Sacramento, CA, 95834, US

(73) Assignee: VORTRAN Medical Technology 1, Inc., Sacramento, CA (US)

(21) Appl. No.: 12/070,154

(22) Filed: Feb. 15, 2008

Related U.S. Application Data
(60) Provisional application No. 60/902,740, filed on Feb. 21, 2007.

(51) Int. Cl. A61M 16/00

(52) U.S. Cl. 128/204.22

(57) ABSTRACT

The present invention pertains generally to a monitoring system for a resuscitator which detects operation of the resuscitator and includes means for informing an operator of function. The monitoring system is triggered by a pressure signal provided by the cycling of the automatic resuscitator from a controlled inhalation phase to a controlled exhalation phase. The monitoring aspect of the system detects specifically a single point low pressure signals which are sequentially compared against an integrated time clock. Failure of the resuscitator system itself to generate a low pressure signal against the integrated time clock causes an alarm condition. Further, gas management is optionally effected by a flow controller integrated into the monitor, a gas management system which responds to the single point low pressure signal and operate a gas valve attached between a gas supply and an automatic resuscitator such that gas is allowed to flow to the resuscitator when the resuscitator is in an inhalation mode and gas flow is interrupted when the resuscitator is in an exhalation mode. The monitoring system includes a low threshold pressure sensor which is actuated by means of a recurrent low pressure pulse generated by the automatic resuscitator itself through the cycling of the resuscitator and remains essentially unaffected by the respiratory cycling of the patient, thus preventing false triggers and greatly simplifying the flow controller operation and format. The low threshold pressure sensor is coupled to a processor wherein the processor reads the occurrence of a pressure event at the pressure sensor and which then closes the gas supply valve and starts a clock. Once the clock reaches a pre-defined duration, the gas supply valve is reopened, the automatic resuscitator continues into an inhalation mode, and the process repeats.

### PARENT CASE TEXT

This application claims the benefit under 35 U.S.C. 119(e) of U.S. provisional applications Ser. No. 60/902,740 filed Feb. 21, 2007, which is incorporated by reference herein in its entirety.

### BACKGROUND

A fundamental aspect of providing respiratory care to a patient is the ability to provide continuous ventilatory support to a patient requiring respiratory assistance. Ventilatory support is typically provided by clinicians and emergency medical personnel through the use of a manual resuscitator or a fully automatic ventilator type device. Decisions as to which device to use is dependent on equipment availability and the personnel resources obtainable to operate the chosen device within necessary functional controls.

Manual resuscitators are generally equipped with a self-inflating bag, a series of check valves, which control the direction of inhalation and exhalation gases, and a patient interface that is either of a nature to fit closely about the patient's nose and mouth or in the alternative, has a port for connecting to an endotracheal tube. Such manual type resuscitators are preferentially connected to a continuous supply of therapeutic gas containing a known percentage of oxygen enrichment. The operator of a manual resuscitator introduces oxygen enriched therapeutic gas into the patient's lungs by applying a constrictive force to the self-inflating bag. As the operator terminates the constrictive force and the self-inflating bag is allowed to refill, pressure of the introduced gas combined with the elastic nature of the patient's own respiratory system causes the introduced gas to then be expelled through the patient's airway and past the check-valves in the manual resuscitator.

Most manual resuscitators are equipped with means to maintain a small minimum positive pressure in the patient's lungs and airways so as to maintain that airway in an “open” condition. This minimal positive pressure is commonly referred to as the “Positive End Expiratory Pressure” or “PEEP”. Upon conclusion of the exhalation phase wherein the patient's respiratory system returns to an ambient pressure, in conjunction with the additional PEEP, the self-inflating bag is again constricted by the operator, the check-valves on the inlet circuit open and the process is repeated.

The ubiquitous practice of manual type resuscitators is evident in the fact that little skill is required to effect cyclic respiration and by the relatively inexpensive nature of such an uncomplicated device. Unfortunately, manual resuscitators can be, and often are, misused and/or misapplied as there is no means within the device for ensuring proper recycle time or appropriate duration of either the inhalation or exhalation phases. A number of studies have been published which show that irrespective of the degree of operator training (as evident in whether the operator of the manual resuscitator is a physician, respiratory therapist, or nurse), patients generally receive volumes of gas per breath, referred to as a “tidal volume”, which are too small and/or are provided to the patient at respiratory rates which are too fast for effective respiration to occur. Inappropriate management of tidal volume has been shown to create significant adverse effects on patients. Representative published journal articles directed to such issues with misuse of manual type resuscitators include “Evaluation of 16 adult disposable manual resuscitators”, Mazzolini D G Jr et al., Respiratory Care. 2004 December; 49(12):1509-14 and “Miss-located pop-off valve can produce airway overpressure in manual resuscitator breathing circuits”, Health Devices. 1996 May-June; 25(5-6):2124, both of which are incorporated by reference in their entireties.

In the alternative to manual type resuscitators, automatic ventilatory devices (often referred to simply as “ventilators”) were originally developed to deliver a set volume of gas to the patient in a set amount of time with little patient monitoring capability by the ventilator itself. In the last twenty-five years different modalities, including pressure control, and significantly enhanced monitoring capabilities have been incorporated as standard elements of the ventilator design. This continuous enhancement and propagation in system capabilities has lead to the creation of the modern transport ventilator.

Transport ventilators generally rely upon a gas volume and time cycled ventilatory mode that operate by delivering to the patient predetermined volumes or constant gas flow for predefined time periods, regardless of the patient's airway/lung compliance. Lung compliance in an emergent-care patient is prone to sudden changes during transport such as resulting from decreased thoracic volume from internal bleeding. Loss of lung compliance in conjunction with application of constant tidal volume by a transport ventilator can cause patient airway pressures to increase to the point that severe injury can occur to the patient. To address the potential patient harm caused by a ventilator, pressure cycled ventilatory and pressure controls have been incorporated within ventilatory support to the patient and further include a number of distinct advantages over straight volume and time cycle ventilatory modalities. Pressure cycled ventilation functions by switching from inhalation to exhalation when a certain pressure is reached regardless of the gas volume supplied. In this later operational mode, the gas volume delivered to the patient varies based on lung compliance, thus preventing the patient from receiving a harmful amount of pressure and insuring appropriate ventilation of the patient.

Modern transport ventilators are battery or pneumatically powered and as aforementioned, are equipped with numerous ventilatory modes, including the pressure cycled operation, various flow control functions, multiple alarm monitoring functions and have the further ability to respond dynamically to the patient allowing for the ventilator to synchronize with the patient breathing efforts. Although current transport ventilators provide consistent, safe, and reliable ventilatory performance, the extreme complexity of the devices result in a very high cost. Additionally, such ventilators require a significant number of disposable accessories with which to operate, the costs associated with the disposable accessories is equivalent to, and often more costly than a complete manual type resuscitator. To reduce high capital investments for the modern ventilator, manufacturers have returned to offering devices with more simplified operational systems focused on time cycled volume modes and without the monitoring, control and alarm features. These devices are often classified as automatic resuscitators and have increase potential for causing patient harm due to dimensioned responsiveness, often cost thousands of dollars to obtain and maintain the requirement for continual outlay of expenditure for disposable support elements.

In today's environment of medical cost containment, hospitals and related medical providers are continuously confronted with limited budgets to procure suitable ventilatory equipment and the required training to properly operate such equipment. Prior attempts to address reduced cost resuscitator equipment having monitoring/flow control attributes have utilized a number of different actions to indicate respiratory response with differing levels of efficiency and effectiveness. U.S. Pat. No. 5,495,848 to Aylsworth et al. utilizes a pressure sensor to determine and proportion gas flow based on degree of inhalation strength. U.S. Pat. No. 6,571,796 to Banner et al. is directed to triggering a gas supply though a demand valve triggered by a drop in tracheal pressure. U.S. Patent Application No. 20060150972 to Mizuta et al. employs an adjusting time scale based on degree of respiratory signal.

The aforementioned monitoring and gas flow controllers have met to a limited degree the functionality requirements needed in a simplified format automatic resuscitator. However, there remains an unmet need for an automatic resuscitator with monitoring and optional gas flow control which requires minimal product knowledge in order to operate safely and provides ventilatory to a patient reliably and reproducibly for extended periods of time.

### SUMMARY OF THE INVENTION

The present invention pertains generally to a monitoring system for a resuscitator which detects operation of the resuscitator and includes means for informing an operator of function. The monitoring system is triggered by a pressure signal provided by the cycling of the automatic resuscitator from a controlled inhalation phase to a controlled exhalation phase. The monitoring aspect of the system detects specifically a single point low pressure signals which are sequentially compared against an integrated time clock. Failure of the resuscitator system itself to generate a low pressure signal against the integrated time clock causes an alarm condition. Further, gas management is optionally effected by a flow controller integrated into the monitor, a gas management system which responds to the single point low pressure signal and operate a gas valve attached between a gas supply and an automatic resuscitator such that gas is allowed to flow to the resuscitator when the resuscitator is in an inhalation mode and gas flow is interrupted when the resuscitator is in an exhalation mode. The monitoring system includes a low threshold pressure sensor which is actuated by means of a recurrent low pressure pulse generated by the automatic resuscitator itself through the cycling of the resuscitator and remains essentially unaffected by the respiratory cycling of the patient, thus preventing false triggers and greatly simplifying the flow controller operation and format. The low threshold pressure sensor is coupled to a processor wherein the processor reads the occurrence of a pressure event at the pressure sensor and which then closes the gas supply valve and starts a clock. Once the clock reaches a pre-defined duration, the gas supply valve is reopened, the automatic resuscitator continues into an inhalation mode, and the process repeats.

In a first embodiment, the processor determines a zero or “off” state, wherein no pressure pulse is presented by the automatic resuscitator, and a triggered or “on” state, wherein a low pressure signal event occurs thus activating the processor. The activated processor compares the on and off states against an integrated time clock and an operator determined cycle time. In the event the time lapse between on and off states exceeds the operator determined cycle time, an alarm condition is triggered.

A further embodiment of the present invention includes a method of controlling gas flow to an automatic resuscitator wherein a pressure sensor detects a low pressure pulse from an automatic resuscitator. The signal from the low pressure sensor is routed to a processor which then adjusts a gas supply valve from a flow-on to a flow-off state. Based on a clock within the processor, once a predefined time is achieved, the gas supply valve is returned to a flow-on state and the automatic resuscitator continues into another inhalation phase.

In a further embodiment, the processor can utilize the clock unit to trigger gas-on and gas-off valve conditions with a delay or advancement of time depending up the detection of a low pressure pulse from the automatic resuscitator. The time duration of the valve being either on or off can also be set to be a fraction or proportion of time wherein the inhalation or gas-on condition and the exhalation or gas-off condition is determined by mathematic division of the time duration from a low pressure signal to a total allowable time, thus creating a ratio of inhalation to exhalation. By using a gas management system in accordance with the present invention, gas supply can be conserved by up to 65% over a system which does not interrupt gas flow.

Further, a monitoring system utilizing a low threshold pressure sensor, a processor, a clock and a gas control valve may be combined directly with an automatic resuscitator so as to provide condition and alarm functions for the overall integrated device. One or more attention attracting devices may be coupled to the processor, such as Light Emitting Diodes (LEDs) or audible alarms can be used.

Other features and advantages of the present invention will become readily apparent from the following detailed description, the accompanying drawings, and the appended claims.

### BRIEF SUMMARY OF THE FIGURES

The invention will be more easily understood by a detailed explanation of the invention including drawings. Accordingly, drawings which are particularly suited for explaining the inventions are attached herewith; however, it should be understood that such drawings are for descriptive purposes only and as thus are not necessarily to scale beyond the measurements provided. The drawings are briefly described as follows:

FIG. 1 is an exploded diagram of a monitoring and gas flow control device in accordance with the present invention.

FIG. 2 is a perspective view of a monitoring and gas flow control device.

FIG. 3 is a left side view of a monitoring and gas flow control device.

FIG. 4 is a right side view of a monitoring and gas flow control device.

FIG. 5 is a front view of a monitoring and gas flow control device.

FIG. 6 is a back view of a monitoring and gas flow control device.

FIG. 7 is a top down view of a monitoring and gas flow control device, particularly showing the control settings and LED alarm elements.

FIG. 8 is bottom up view of a monitoring and gas flow control device, particularly showing the sensor port for detecting a low pressure signal from the exhaust of an adjoining modulator.

FIG. 9 is a perspective view of a monitoring and gas flow control device proximal to the modulator of an automatic resuscitator.

FIG. 10 is a perspective view of a monitoring and gas flow control device affixed to the modulator of an automatic resuscitator such that the sensor port of the device is in fluid communication with the sample port of the adjoining modulator.

FIG. 11 is a cross sectional diagram of a modulator from an exemplar automatic resuscitator wherein the modulator is in an inhalation mode.

FIG. 12 is a cross sectional diagram of a modulator from an exemplar automatic resuscitator wherein the modulator is in an exhalation mode and a low pressure pulse has been generated in the sample port.

FIG. 13 is a diagram of a monitor/gas flow control in a gas supply loop and connected to a patient.

### DETAILED DESCRIPTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

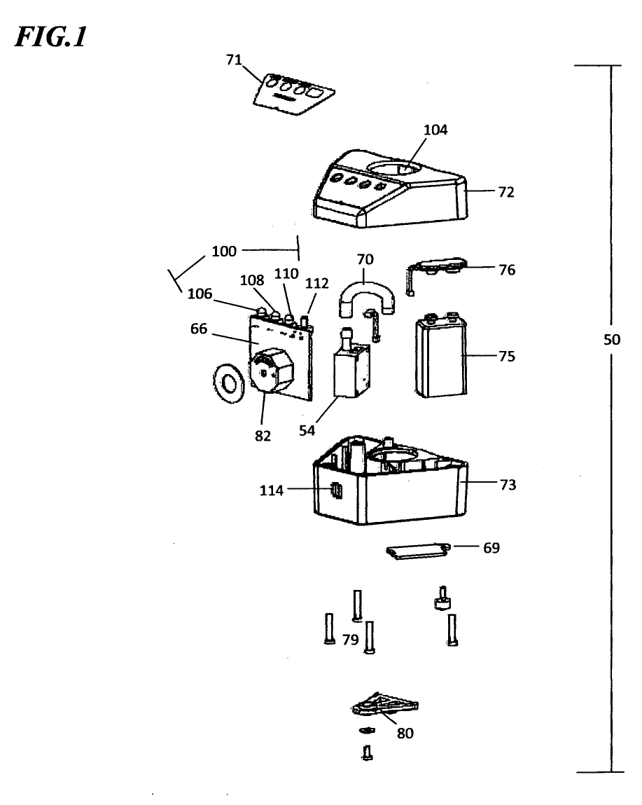

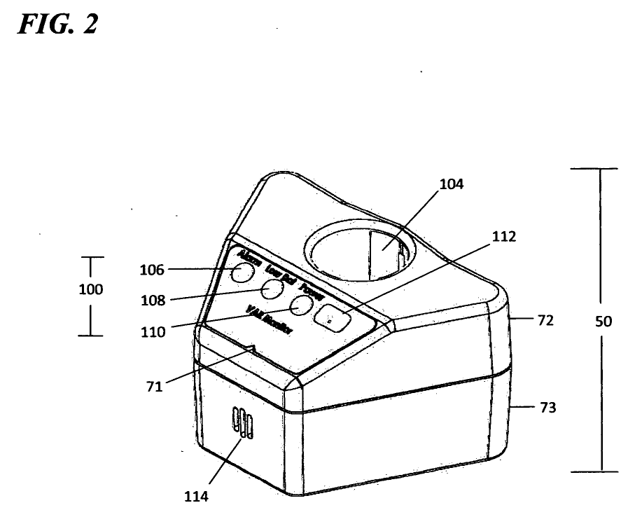

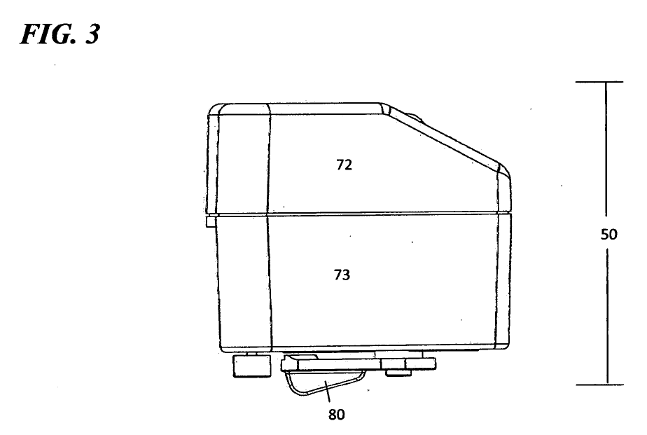

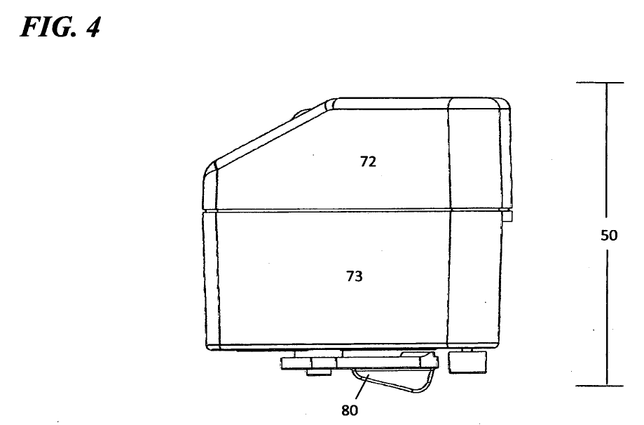

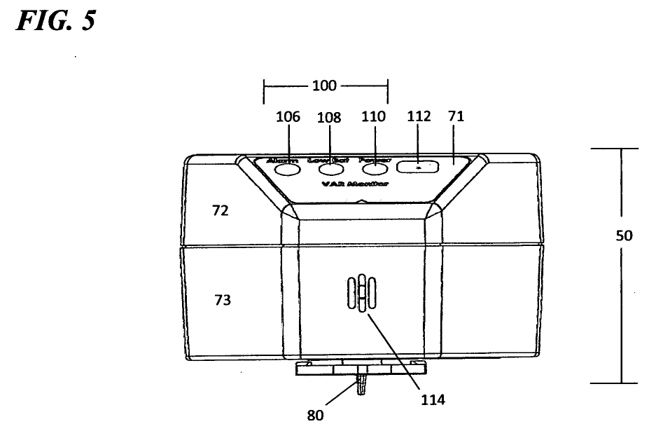

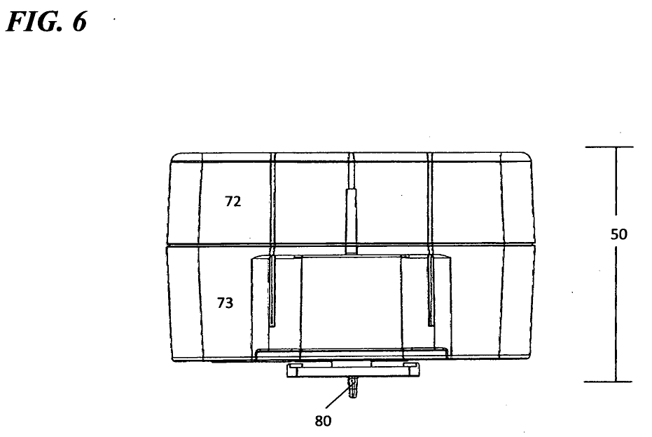

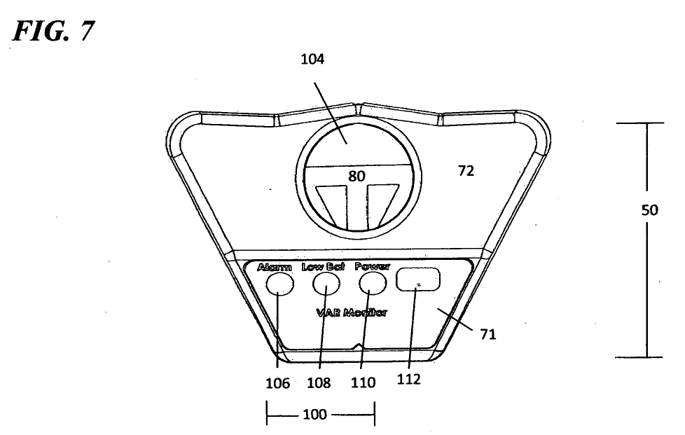

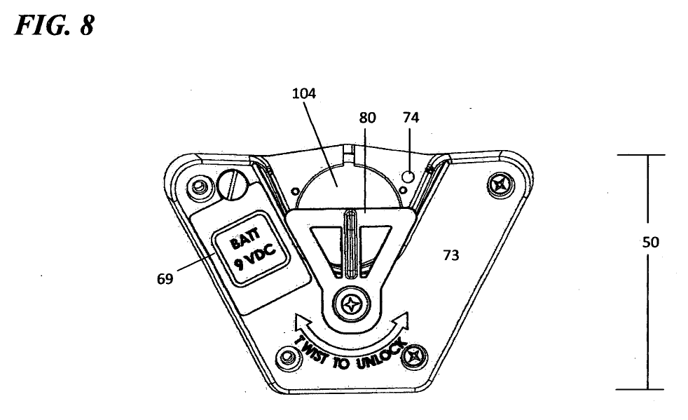

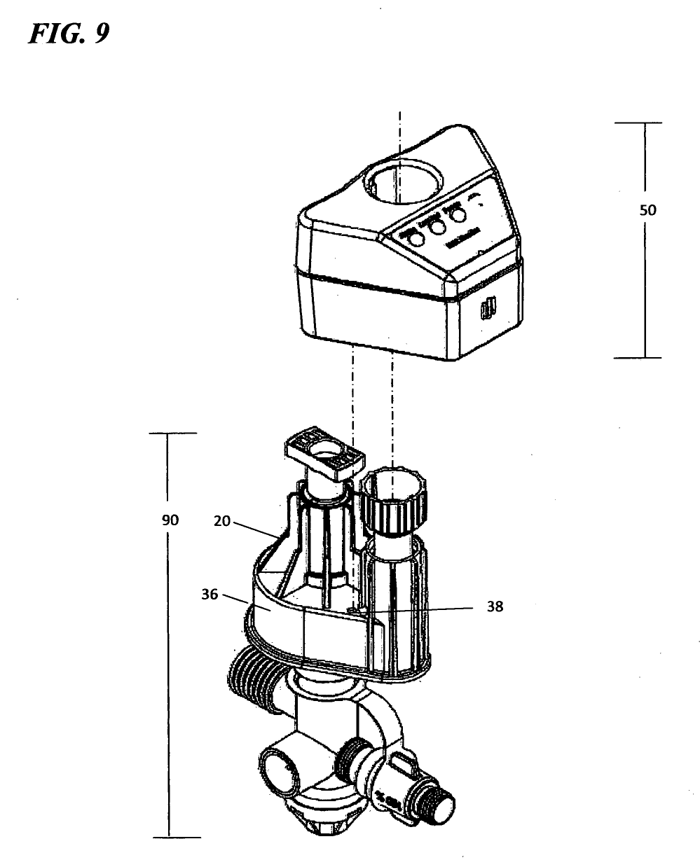

Referring more specifically to the figures, for illustrative purposes the present invention is embodied in the apparatus generally shown in FIG. 1 through FIG. 13.

FIG. 1 illustrates a monitor/gas flow controller 50. The unit is comprised of upper housing case 72 and lower housing case 73. Functional components include pressure sensor 54 in fluid communication with line 70 and sample port 74, processor 66 with associated alarm component (herein shown as LED's 106, 108, and 110), a power source 75 coupled to processor 66 by way of power connection 76. On upper case 72 is face plate 71. Face plate 71 includes indicia for the coinciding alarm component LED's 106, 108 and 110 so as to provide a means for the operator to determine the type or nature of alarm or status code. Buzzer 114 is located in the case, and specific to embodiment shown, within lower housing case 73. Upper case 72 and lower housing case 73 are engaged upon one another to effect closure and entrainment of the functional elements of the monitor/gas flow controller within the protective environment created therein. Upper housing case 72 and lower housing case 73 may be maintained in a durable connection through suitable mechanical engagement, include snap fit, adhesive and threaded fastener (threaded screws 79 are shown in FIG. 1 as an exemplary means of engagement). A power source door 69 is included by which power source 75 can be replaced as needed. An on/off switch may be provided as button 112

FIG. 1 further depicts capture cam 80. Capture cam 80 is rotatably engaged into lower housing case 73 such that the arc of movement by capture cam 80 allows for the monitor/gas flow controller 50 to be engaged directly onto an appropriately configured automatic resuscitator 90 and retain the monitor/gas flow controller 50 onto automatic resuscitator 90.

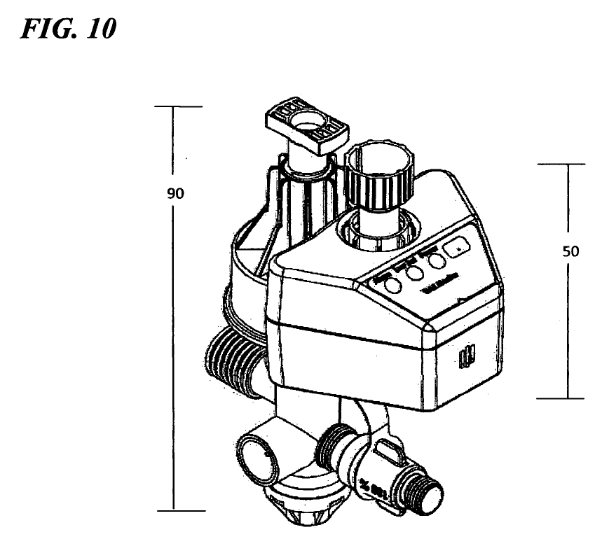

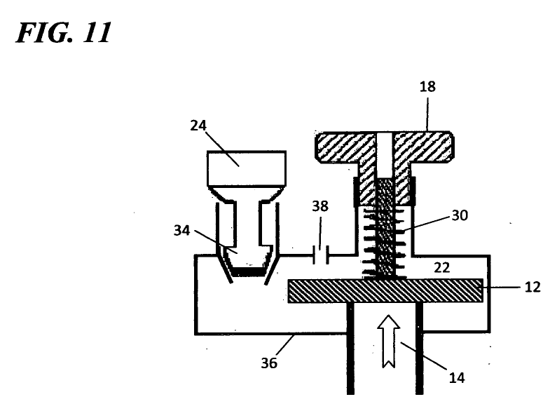

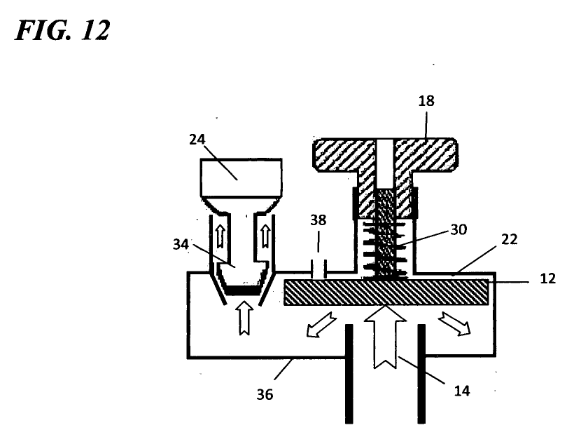

FIG. 10 depicts a monitor/gas flow controller 50 and an automatic resuscitator 90. It is appreciated that flow controller 50 may be a standalone device with a separate fluid communication with the automatic resuscitator 90, or may be directly integrated with automatic resuscitator 90. Further details for the automatic resuscitator 90, as illustrated in FIGS. 11 and 12, may be found with reference to commonly owned U.S. Pat. No. 6,067,984, herein incorporated by reference in its entirety. Although the specific embodiment detailed with respect to FIGS. 10 to 12 detail an exemplary automatic resuscitator, it is appreciated that the gas flow controller, monitor and alarm of the present invention may be coupled to or integrated with any compatible resuscitator having the ability to generate a low pressure pulse by action of a exhalation triggered event by the resuscitator.

The automatic resuscitator 90 includes a modulator 20, which operates as a valve that opens at one pressure and closes at a second lower pressure when connected to a pneumatic capacitor. A pneumatic capacitor may comprise anything that increases in volume with an increase in pressure. For purposes of the present invention, the patient's own lungs 130 generally act as that pneumatic capacitor.

FIGS. 11 and 12 schematically illustrate modulator 20 in the capacity of a ventilatory support. The primary actuating mechanism of the modulator 20 is piston 12. Piston 12 is bias loaded by spring 30, and has adjustment means (i.e. pressure dial 18) which operates in a similar fashion to a pop-off valve for releasing internal pressure once a defined threshold is exceeded.

Piston 12 is coupled to a patient's airway via inlet port 14. The area of piston 12 that is exposed to the patient's airway pressure, and thus the pressure across the face of piston 12, varies depending on whether the piston is in an open or closed position.

FIG. 12 illustrates modulator 20 with piston 12 in the open or exhalation position. In this configuration, the full face of the piston is exposed to the patient's airway pressure (i.e. the exposed area is a function of the diameter of piston 12). The force of the patient's airway pressure on the piston face 16 is the product of the patient's airway pressure and the exposed area of the piston. For any given setting of pressure dial 18, the force of spring 30, is the same when the piston is just opening or just closing. Since the force of spring 30 is the only force resisting the patient's airway pressure on piston 12, piston 12 will open at a much higher pressure than when it closes.

In the closed inhalation position (FIG. 11) the area of the piston 12 exposed to the Patient's airway pressure is the area circumscribed by the inlet column 14 in contact with the piston (which is smaller than the area of piston face 12).

The representative automatic resuscitator 90 operates utilizing compressed gas. When piston 12 in modulator 20 is in a closed position, gas flow from gas supply 52 is directed to the patient and the pressure against piston 12 rises as inhalation continues. During this stage of the resuscitator's inhalation cycle, opposite side 22 of piston 12 (i.e. the pressure inside the modulator housing) is at a lower pressure. When a set peak inhalation pressure (PIP) is reached, piston 12 opens and exhausts the inhaled gases (FIG. 12) This momentarily increases the pressure inside the modulator housing (opposite side 22) as the exhaled gas is released through exhalation resistance valve 24 and causes a low pressure pulse through signal port 38.

This phenomenon of changing pressures inside the modulator housing during the transition from inhalation to exhalation creates a “low pressure signal” that triggers processor 66. The “low pressure signal” provides a triggering condition in pressure sensor 54 (i.e. pressure sensitive or diaphragm type switch) and a subsequent electrical on signal is generated. Pressure sensor 54 preferably comprises a pneumatic pressure sensor, and it has a threshold sensitivity of approximately 0.5 cm-water. The operating temperature range of sensor 54 as provided above is in the range of −40° F. to 205° F. Sensor 54 is coupled to modulator 20 via line 70 that is in fluid communication with sample port 74 in lower housing case 73 and modulator 20 through signal port 38 in modulator housing 36.

The use of a “low pressure signal” is unique to modulator 20 as this signal specifically signifies the resuscitator is cycling from inhalation to exhalation with a slight shift in pressure. Based on the knowledge of the pressure changes in the automatic resuscitator's modulator 20, a number of functions can be applied. For example, the signal may be used to allow flow controller 50 to turn off the gas flow during exhalation for a pre-determined period of time. Additionally, the signal may be used for triggering an alarm condition when there is a failure to cycle and thus providing warning if the modulator is not cycling, and thereby patient resuscitation has stopped.

Referring to FIG. 1, monitor/gas flow controller 50 comprises a printed circuit board (PCB) 62 having a processor 66 configured to receive input regarding low pressure signals from pressure sensor 54, and use of that signal to either turn on or turn off gas flow from the gas supply to the automatic resuscitator. Thus gas flow controller facilitates conservation of the amount of therapeutic gas supplied to the automatic resuscitator by providing gas only during the inhalation phase by the automatic resuscitator 90. The exhalation time may be defined by a clock function against timer function embedded in printed circuit board 62 such that upon reaching an operator defined time, the monitor/gas flow controller 50 turns the gas flow on. Further, the processor may divide the triggered time against a maximum cycle time entered by the operator, such a ratio of inhalation to exhalation can occur and the gas flow controller is operated accordingly.

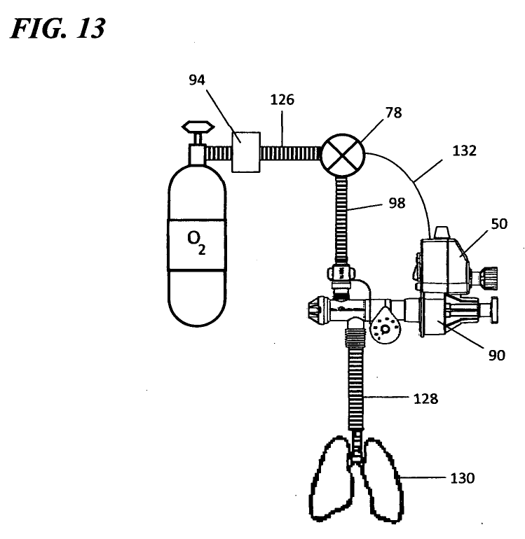

FIG. 13 illustrates monitor/gas flow controller 50, automatic resuscitator 90 and gas supply 52. In this configuration, gas flows through flow meter 94 and gas supply line 126 into controller primary gas control valve 78. Primary gas control valve 78 allows for distribution of gas into resuscitator input line 98 and conversely into automatic resuscitator 90. For proper operation, the resuscitator supply line should provide automatic resuscitator 90 gas in the flow range of at most 40 liters a minute. In this figure, monitor/gas flow controller 50 is integrated within a monitor housing such that the housing also functions in retaining an alarm.

Processor 66 uses the signal generated by pressure sensor 54 and based on a reading of a low pressure signal, sends a signal via cable 132 to primary gas control valve 78, which may be either a solenoid type valve or common supply gas flow meter to cause the opening and closing of the primary gas control valve and thus regulate the flow of gas from gas supply 52. The primary gas control valve 78 is generally an open type valve and uses sufficient voltage to cause the valve to close. In the event of a power failure, primary gas control valve 78 stays open and permits gas flow from gas supply 52 and gas supply line 126 through primary gas control valve 78 into resuscitator input line 98 and to the automatic resuscitator 90.

The monitor/gas flow controller 50 is configured to control gas flow such that the gas flow into the automatic resuscitator 90 is stopped during exhalation. This is particularly beneficial in extending the automatic resuscitator 90 operation time when supplied gas is limited by the amount of available compressed gas (oxygen or air), particularly in the event of an emergency. This feature conserves gas and increases operational periods by as much as 300%.

The monitor/gas flow controller 50 may also be configured with a time controller embedded in circuit board 62 of processor 66 which operates via an electric signal to operator determined exhalation time of the automatic resuscitator 90. For example, the timer may be used to set the exhalation time from a range of settings (i.e. from approximately 0.5 second to over 6 seconds). The timer may be set through a touch button interface (such as cycling of button 112 from off to different time settings as an “on” condition or, in the alternative, an optional timer selection knob (not shown) allows an operator to set the desired exhalation time or an inhalation to exhalation ratio.

Referring specifically to FIGS. 1 to 12, a non-cycling alarm monitor 106 is shown for use with automatic resuscitator 90 and modulator 20. The monitor preferably is embedded in upper housing case 72 and lower housing case 73 wherein lower housing case 73 is configured to connect to modulator 20. The housing has an aperture 104 to allow monitor/flow controller 50 to be positioned over and around exhalation resistance valve 24 and against modulator 20. It is appreciated that housings 72 and 73 may comprise any number of shapes and contours to interface with a corresponding resuscitator. In the alternative, monitor/flow controller 50 may be a standalone device which cooperatively integrates with any number of different resuscitator devices. Preferably, the monitor/flow controller 50 is configured to be packaged as a small portable footprint which can be efficiently used with automatic resuscitator 90 in emergency situations.

Monitor/flow controller 50 may comprise only a monitor function comprising processor 66 that analyzes a low pressure signal event from pressure sensor 122 to test for a non-cycling condition of the automatic resuscitator. This condition can then trigger an alarm event to notify attending personnel of an abnormal condition, such as non-cycling or the like. The alarm may comprise a low power alarm with visual and/or audible warning when the automatic resuscitator 90 stops cycling.

In a preferred embodiment, the monitor upper housing case 72 may be configured to hold a plurality of light emitting diodes (LED's) 106, 108, and 110, each of which is coupled to processor 66. A first LED 110 may emit light of a first color (e.g. yellow) to indicate the cycling of breathing. LED 110 may be configured to stay on during exhalation and to remain off during inhalation time. A second LED 108 having a second color (e.g. green) may show that the overall system is on and has sufficient power to operate. A third LED 106 having a third color (e.g. red), may be used to show an alarm condition. In normal operation, the third LED 106 stays in an off condition. However, if there is a power failure or the device stops cycling, the third LED 106 comes on.

The monitor/gas flow controller unit 50 is powered upon activating an On/Off (I/O) switch 112. Once monitor/gas flow controller unit 50 is turned on, the system goes to a power-on test mode. At his point, the processor 66 may be configured to turn on LED's and buzzer 114 for a one second period to the test the device's operational readiness. Monitor/gas flow controller unit 50 may also indicate a low battery condition with LED 106 showing yellow. During this time, the processor 66 may check the battery voltage, and control LED 106 to blink if the battery voltage is less than nominal voltage (i.e. to blink when 5.5 VDC are available in a 9.0 VDC system).

While powered-on, processor 66 monitors the pressure sensor 54 for a low pressure signal. If a low pressure signal does not occur after a predetermined time set by the operator or attending personnel, such as an eight (8) second period, a failure mode is detected and an alarm is activated. A blinking LED 106 may be used to indicate a non-cycling condition. The alarm will remain on until the failure condition is corrected and a low pressure signal is provided by operation of the automatic resuscitator 90. During operation of monitor/gas flow controller unit 50, the monitor will indicate a power-on mode by illumination of LED 108. LED 110 may blink or flash (turn off momentarily) when a low pressure signal is detected by cycling of the automatic respirator from inhalation to exhalation mode.

The pressure sensor 54 generally has minimum detectable pressure change of 0.5 cm-water. Optionally, when the alarm is in a ready state, the algorithm contained in the logic of processor 66 will check for a low pressure signal and time from a clock function. If no low pressure signal is detected after a finite period of time (e.g. 8 seconds elapsed), both LED 110 and buzzer 114 may be triggered as part of the alarm condition. Preferably, alarm buzzer exhibits a loudness of 75 dB at one (1) meter distance from the device when enclosed, or a 70 dB rating at one (1) meter if the buzzer is not enclosed. Both LED 110 and buzzer 114 may stay on until the error is corrected by an operator, or the main power switch 116 is turned off. If the error condition is remedied, the alarm will reset and the combined LED/buzzer will turn off.

The general construction of functional elements of monitor/gas flow controller unit 50, as well as casing and control surfaces, may comprise polymer, nonferrous or ferrous compositions. Preferably, the functional elements are fabricated from suitable medical service, oxygen rated materials such as K-resin and ABS plastics.

### EXAMPLE

A monitor with gas flow control was fabricated in accordance with the present invention.

Upon testing, the device was routinely capable of maintaining operation under the following conditions:

Peak Inhalation Pressure Range: 10 to 50 cm-water

Gas Flow Rates: Up to and including 40 liters per minute

Maximum Gas Supply Pressure: 50 PSI

Operation Time under Continuous Duty: >72 hrs

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

1. A device for monitoring an automatic resuscitator, comprising
a. an automatic resuscitator device which creates a low pressure signal upon cycling of the automatic resuscitator from an inhalation mode to an exhalation mode;
b. a pressure sensor;
c. a processor unit coupled to said pressure sensor;
d. a time function having a user defined duration;
e. an alarm means;
wherein said pressure sensor is in communication with the automatic resuscitator and is configured to detect a low pressure signal generated by the automatic resuscitator;
wherein said processor unit detects a low pressure signal through said pressure sensor and upon detection of a low pressure signal, said time function is initiated;
wherein if a subsequent low pressure signal does not occur within the user defined duration set in the time function, an alarm means is triggered.

2. A device as in claim 1, wherein low pressure signal is at least 0.5 cm-water.

3. A device as in claim 1, wherein the user defined duration is greater than or equal to one half of one second (0.5) and less than or equal to six (6) seconds.

4. A device for controlling gas flow to an automatic resuscitator, comprising
a. An automatic resuscitator device which creates a low pressure signal upon cycling of the automatic resuscitator from an inhalation mode to an exhalation mode;
b. A pressure sensor;
c. A processor unit coupled to said pressure sensor;
d. A gas flow controller connected to said automatic resuscitator and said processor unit;
e. A gas supply source connected to said gas flow controller;
wherein said pressure sensor is in communication with the automatic resuscitator and is configured to detect a low pressure signal generated by the automatic resuscitator;
wherein said processor unit detects a low pressure signal through said pressure sensor and sends a control signal to the gas flow controller whereby the gas flow controller stops flow of gas from the gas supply to the automatic resuscitator for a predetermined length of time; and
wherein upon expiration of the predetermined length of time, the gas flow controller then continues supply of gas to the automatic resuscitator and the process is allowed to repeat.

5. A device as in claim 4, wherein low pressure signal is at least 0.5 cm-water.

6. A device as in claim 4, wherein the gas flow is stopped for a duration of greater than or equal to one half of one second (0.5) and less than or equal to six (6) seconds.

7. A device as in claim 4, wherein the control signal turns off the gas flow equivalent in duration to between 10% and 90% of a patient exhalation period.

8. A device as in claim 4, wherein the control signal turns off the gas flow equivalent in duration to between 10% and 50% of a period defined by a total inhalation/exhalation period.

9. A device as in claim 4, wherein the processor is configured to analyze said low pressure signal to determine a failure to cycle after a finite period of time.

10. A device as in claim 9, wherein if there is a failure to cycle after a finite period of time, an alarm condition is triggered.

11. A device as in claim 10, wherein the alarm condition is visual, auditory or the combination thereof.

12. An automatic resuscitator, comprising
a. A modulator which creates a low pressure signal upon cycling of the automatic resuscitator from an inhalation mode to an exhalation mode in respiratory support of a patient;
b. A pressure sensor;
c. A processor unit coupled to said pressure sensor;
d. A gas flow controller connected to said automatic resuscitator and said processor unit;
e. A gas supply source connected to said gas flow controller;
wherein said pressure sensor is in communication with the modulator and is configured to detect a low pressure signal generated by the modulator;
wherein said processor unit detects a low pressure signal through said pressure sensor and sends a control signal to the gas flow controller whereby the gas flow controller stops flow of gas from the gas supply to the modulator for a predetermined length of time; and
wherein upon expiration of the predetermined length of time, the gas flow controller then continues supply of gas to the modulator and the process is allowed to repeat.

13. An automatic resuscitator as in claim 12, wherein low pressure signal is at least 0.5 cm-water.

14. An automatic resuscitator as in claim 12, wherein the gas flow is stopped for a duration of between 0.5 second and 6 seconds.

15. An automatic resuscitator as in claim 12, wherein the control signal turns off the gas flow equivalent in duration to between 10% and 90% of a patient exhalation period.

16. An automatic resuscitator as in claim 12, wherein the control signal turns off the gas flow equivalent in duration to between 10% and 50% of a period defined by a total inhalation/exhalation period.

17. An automatic resuscitator as in claim 12, wherein the processor is configured to analyze said low pressure signal to determine a failure to cycle after a finite period of time.

18. An automatic resuscitator as in claim 17, wherein if there is a failure to cycle after a finite period of time, an alarm condition is triggered.

19. An automatic resuscitator as in claim 18, wherein the alarm condition is visual, auditory or the combination thereof.

20. A method for controlling gas flow to an automatic resuscitator, comprising;
a. sensing a low pressure event inside an exhalation chamber of an automatic resuscitator;
b. generating a control signal based on the low pressure event inside said exhalation chamber;
c. stopping flow of gas to said automatic resuscitator as a function of the control signal; and
d. returning the flow of gas to said automatic resuscitator after expiration of a finite period of time.

21. A method as in claim 20, wherein low pressure signal is at least 0.5 cm-water.

22. A method as in claim 20, wherein the gas flow is stopped for a duration of between 0.5 second and 6 seconds.

23. A method as in claim 20, wherein the control signal turns off the gas flow equivalent in duration to between 10% and 90% of a patient exhalation period.

24. A method as in claim 20, wherein the control signal turns off the gas flow equivalent in duration to between 10% and 50% of a period defined by a total inhalation/exhalation period.

25. A method as in claim 20, wherein failure of a control signal to occur after a finite period of time triggers an alarm condition.

26. A method as in claim 25, wherein the alarm condition is visual, auditory or the combination thereof.
